(19) United States
(12) Patent Application Publication
(10) Pub. No.: US 2005/0187468 A1
(43) Pub. Date: Aug. 25, 2005

(54) Modified esophageal doppler monitor methods for measuring aortic dp/dt and pulse wave velocity

(76) Inventor: Glen M Atlas, Livingston, NJ (US)

Correspondence Address: Licata & Tyrrell, 66 East Main Street, Marlton, NJ, 08053, US

(21) Appl. No.: 10/497,056

(22) PCT Filed: Nov. 26, 2002
(86) PCT No.: PCT/US02/38057

Related U.S. Application Data
(60) Provisional application No. 60/338,557, filed on Nov. 29, 2001.

(51) Int. Cl. A61B 8/00

(52) U.S. Cl. 600/437

(57) ABSTRACT

A modified esophageal Doppler monitor with two ultrasonic Doppler transducers that is capable of measuring time rate change of pressure within the aorta and aortic pulse wave velocity is provided. Methods of using this modified monitor to assess aortic compliance, diagnose atherosclerotic disease and assess efficacy of therapeutic agents for atherosclerotic disease are also provided.

This application claims the benefit of priority from U.S. Provisional Application Ser. No. 60/338,557, filed Nov. 29, 2001, which is herein incorporated by reference in its entirety.

### FIELD OF THE INVENTION

The present invention relates to a modified esophageal Doppler monitor capable of measuring time rate change of pressure and pulse wave velocity in the aorta of a subject. Measurement of these parameters is useful in assessing aortic compliance and/or distensibility, important risk factors in subjects with aortic dissections and aortic aneurysms. Measurement of aortic compliance in a subject is also useful in diagnosing atherosclerotic disease and assessing efficacy of therapeutic agents for atherosclerotic disease.

### BACKGROUND OF THE INVENTION

Esophageal Doppler monitors (EDMs) are known and have been described, most recently, in U.S. Pat. No. 4,796,634. The EDM of U.S. Pat. No. 4,79,6,634 is designed to predictively calculate the area of a patient's ascending aorta from his or her age, sex, height and weight; provide a signal indicative of the systolic velocity of the blood flowing through the patients descending aorta; convert or scale that signal to one representative of the velocity of the blood flowing through the patient's ascending aorta; and compute the patient's cardiac output from the calculated aortic diameter and the scaled up velocity signal. Accordingly, this EDM can be used to measure cardiac output, afterload, preload, peak velocity and acceleration of aortic blood flow.

The EDM of U.S. Pat. No. 4,796,634 consists of means inclusive of an esophageal probe for measuring the systolic velocity of blood flowing through a patient's aorta, a means for calculating the cross-sectional area of a patient's aorta and a means for calculating the cardiac output of the patient from the systolic flow velocity and the aortic diameter. The esophageal probe of the EDM taught in U.S. Pat. No. 4,796,634 consists of an esophageal stethoscope with an ultrasonic transducer tip mounted on the lower end of the stethoscope.

The EDM of U.S. Pat. No. 4,796,634 has several advantages over traditional ultrasound devices and pulmonary artery catheters in that it is less expensive, easier to use, and offers continuous real-time measurement of both peak velocity and mean acceleration of descending aortic blood flow (Singer, M. Int. Anesthesiol. Clin. 1993 31:99-125). For intubated patients, this EDM can readily be placed orally and used both intraoperatively and postoperatively without difficulty. Nasal placement in conscious patients has also been described (Atlas, G. and Mort, T. Chest 2001 119:319).

In the present invention a modified EDM is provided which, in addition to measuring cardiac output, afterload, preload, peak velocity and acceleration of aortic blood, can also be used to measure time rate change of pressure within the aorta (aortic dP/dt) and aortic pulse wave velocity.

### SUMMARY OF THE INVENTION

An object of the present invention is to provide a modified esophageal Doppler monitor capable of measuring time rate change of pressure within the aorta (aortic dP/dt) and aortic pulse wave velocity as well as cardiac output, afterload, preload, peak velocity and acceleration of aortic blood flow. The modified esophageal Doppler monitor of the present invention comprises an esophageal probe with two ultrasonic Doppler transducers separated by a known distance so that peak velocities of aortic blood flow can be measured a two different points of the descending aorta.

Another object of the present invention is to provide a method for measuring aortic pulse wave velocity in a subject comprising inserting into the esophagus of a subject an esophageal probe of a modified esophageal Doppler monitor having two ultrasonic Doppler transducers separated by a known distance; measuring time between peak velocities detected as each ultrasonic Doppler transducer; and calculating the aortic pulse wave velocity in the subject.

Another object of the present invention is to provide a method for assessing aortic compliance and/or distensibility in a subject comprising inserting into the esophagus of a subject an esophageal probe of a modified esophageal Doppler monitor having two ultrasonic Doppler transducers separated by a known distance and determining aortic wave pulse velocity and time rate change of pressure within the aorta (aortic dP/dt) in the subject so that compliance and/or distensibility of the aorta in the subject can be assessed. Assessment of compliance of the aorta in accordance with the method of the present invention is useful in detection of atherosclerotic disease in the subject.

Yet another object of the present invention is to provide a method of assessing efficacy of therapeutic agents on modulating compliance of the aorta in a subject. In this method, compliance of the aorta is first assessed using a modified esophageal Doppler monitor of the present invention. The subject is then administered a therapeutic agent for a selected time adequate to be therapeutically effective and compliance of the aorta in the subject is reassessed using the modified esophageal Doppler monitor. A change in compliance of the aorta after administration of the therapeutic agent as compared to compliance of the aorta prior to administration of the therapeutic agent is indicative of the therapeutic agent being effective at modulating compliance of the aorta. Agents useful against atherosclerotic disease are expected to increase aortic compliance. This method is particularly useful in assessing efficacy of therapeutic agents such as beta-blockers and other anti-hypertensive agents at increasing the compliance of the aorta.

### BRIEF DESCRIPTION OF THE FIGURES

FIG. 1 is a diagram of a modified esophageal Doppler monitor showing the two transducers and the esophageal stethoscope of the esophageal probe.

FIG. 2 is a diagram depicting placement of the esophageal probe of the modified esophageal Doppler monitor within the esophagus of a subject. FIG. 2.

FIG. 3 is a diagram illustrating aortic blood flow velocity versus time. Aortic blood flow acceleration, dV/dt, is the slope of this curve.

FIG. 4 is a block diagram depicting the esophageal Doppler monitoring technique for measuring pulse wave velocity.

### DETAILED DESCRIPTION OF THE INVENTION

The present invention relates to a modified esophageal Doppler monitor capable of measuring time rate change of pressure (aortic dP/dt) and pulse wave velocity (V_{pw}) in the aorta of a subject as well as cardiac output, afterload, preload, peak velocity and acceleration of aortic blood. This new device is useful for cardiovascular monitoring. In particular, the device is useful for medical and surgical management of patients with acute aortic injury.

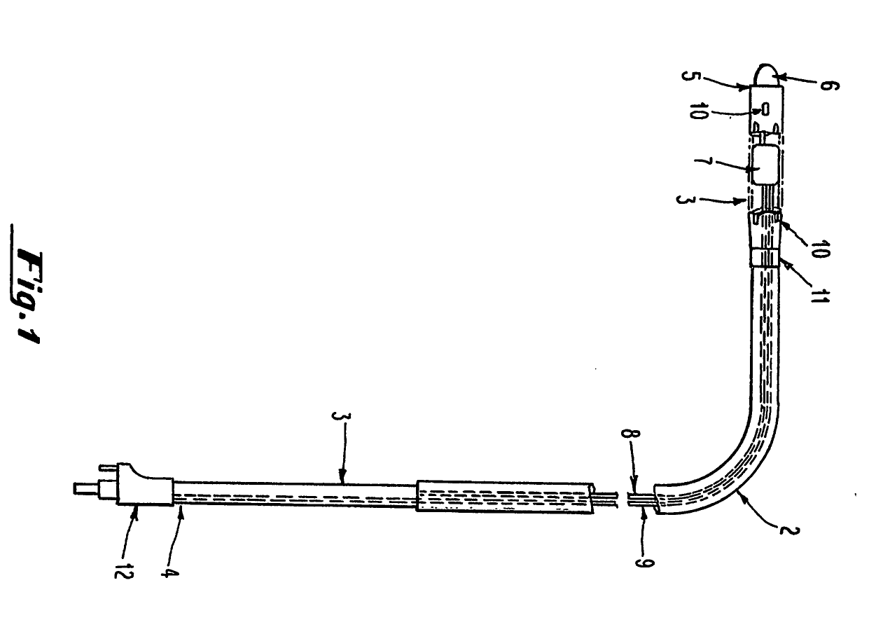

As depicted in FIG. 1, the modified esophageal Doppler monitor of the present invention comprises an esophageal probe 2. The esophageal probe 2 comprises an esophageal stethoscope 3 with an upper end 4 and a lower end 5 and a first 6 and second 7 ultrasonic Doppler transducer mounted in the esophageal stethoscope 3.

The esophageal stethoscope is a flexible hollow tube preferably equipped with acoustical ports 10 in its lower end 5. When equipped with acoustical ports, an acoustically transparent sleeve 11 is sealed to the tube around the ports to prevent mucous and other substances in the esophagus from clogging the acoustical ports. In this preferred embodiment, an acoustical coupling 12 is fitted to the upper end of the esophageal stethoscope so that the esophageal stethoscope can be connected to a standard physician's listening device so that the physician may listen to the subject's breathing, heart beat, etc. The esophageal stethoscope may further comprise a thermistor at its lower end for monitoring of a subject's body temperature.

The first ultrasonic Doppler transducer 6 is mounted in the lower end 5 of the esophageal stethoscope 3. The second ultrasonic Doppler transducer 7 is mounted in the esophageal stethoscope 3 at a selected known distance, preferably about 2 to about 5 centimeters, more preferably about 3 to about 4 centimeters from the first ultrasonic Doppler transducer 6 between the upper 4 and lower end 5 of the esophageal stethoscope 3. Each ultrasonic Doppler transducer comprises a receiver and a transmitter. Each receiver detects the frequency shift associated with the change in velocity within the aorta at its position and the transmitter transmits the frequency shift to a means located outside of the subject for determining the aortic pulse wave velocity in the subject. Exemplary ultrasonic Doppler transducers useful in the present invention include those set forth in U.S. Pat. No. 4,796,634, the teachings of which are herein incorporated by reference in their entirety.

Various means for determining aortic pulse wave velocity can be incorporated into the esophageal Doppler monitor. In one embodiment, wires connect the first and second ultrasonic Doppler transducer to voltage converters located outside the subject. As depicted in FIG. 1, these wires 8, 9 are positioned inside the esophageal stethoscope and extend up from each transducer to the upper end of the esophageal stethoscope wherein they are connected to voltage converters. The voltage converter then transmits the velocity from each transducer to a peak detector for each transducer, also located outside the subject. Each peak detector then transmits its signal of detected maximal velocity change to a high-speed timer, which then transmits the time between detected peak velocity changes at each transducer to a high-speed timer. The high-speed timer transmits the time between detected peak velocity changes to a microcomputer, which then calculates aortic pulse wave velocity by dividing the known distance between the ultrasonic Doppler transducers by the measured time between detected maximal velocity changes.

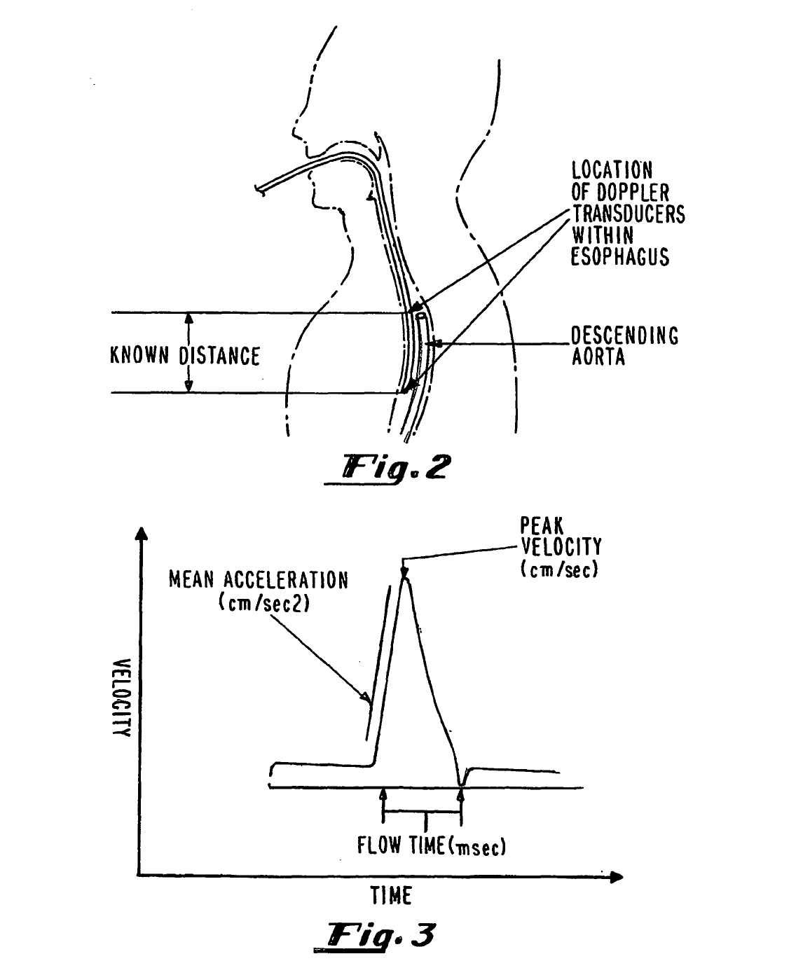

The esophageal stethoscope of the esophageal probe is inserted via its lower end into the subject's mouth, typically after he or she is anesthetized, and passed downward through the subject's esophagus until the first ultrasonic Doppler transducer mounted at the lower end of the esophageal stethoscope and the second ultrasonic Doppler transducer mounted a selected distance from the first transducer between the upper and lower end of the esophageal stethoscope are both positioned opposite to a region of the subject's descending aorta. See FIG. 2.

Measurement by the esophageal Doppler monitor of the present invention is based upon a wave transmission model using the following equation of continuity (Milnor WR. Hemodynamics. Baltimore: Williams & Wilkens 1982:20-22):
dP/dt=ρ·V²_{pw} (dV/dx) Equation (1)
wherein
V²_{pw}=(dx/dt)² Equation (2)
Substituting Equation (2) into Equation (1) yields:
dP/dt=ρ·(dx/dt)²·dV/dx=ρ·(dx/dt)·dV/dt Equation (3)
Thus:
dP/dt=ρ·V_{pw}·(dV/dt) Equation (4)

The modified esophageal Doppler monitor of the present invention equipped with two ultrasonic Doppler transducers separated by a known distance, x, within the esophagus can be used to measure the aortic pulse wave velocity, V_{pw}. V_{pw} is determined from Equation 5 as follows:

$${V}_{pw} = \frac{distance between Doppler transducers}{time between detected peak velocities} \qquad Equation ( 5 )$$

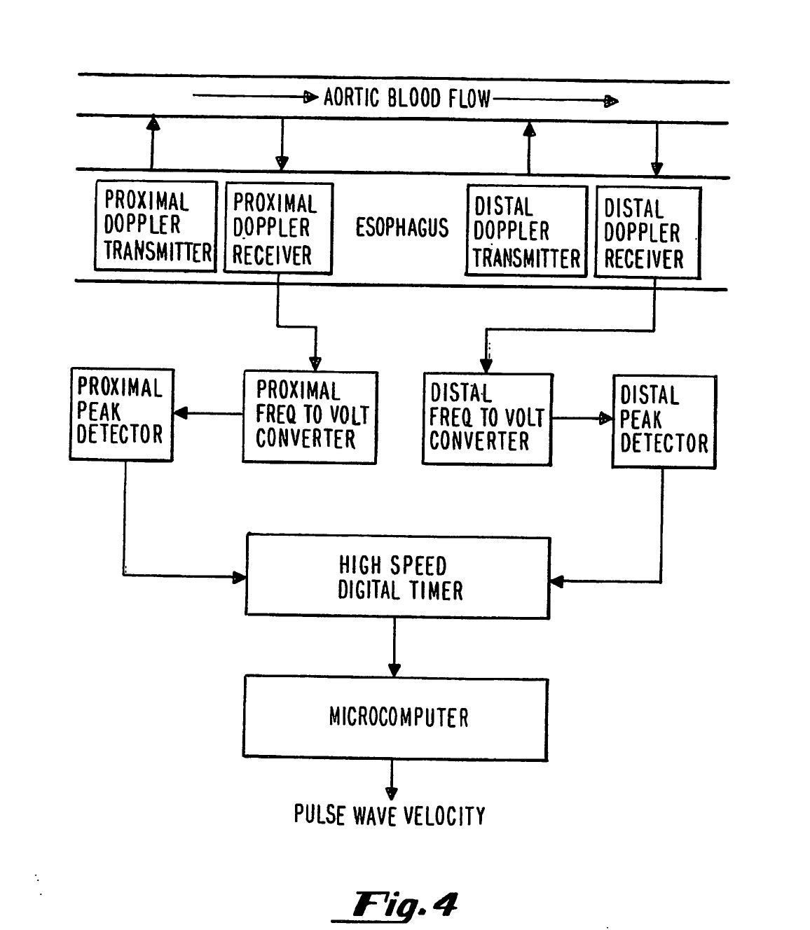

FIG. 4 is a block diagram illustrating the implementation of this technique.

As will be understood by those of skill in the art upon reading this disclosure, these equations are applicable to any measurable wave velocity. Peak velocity has been used simply for exemplary purposes. However, initial wave velocities, ending wave velocities, etc. can also be measured at each transducer and used in these equations to determine V_{pw} and aortic dP/dt.

The time rate change of pressure within the aorta (aortic dP/dt) can then be determined from Equation 4 as shown above. In this equation, dV/dt is the acceleration of aortic blood flow. This is readily determined using the modified esophageal Doppler monitor of the present invention or that described in, for example U.S. Pat. No. 4,796,634, by taking the first derivative with respect to time of aortic blood flow velocity. This is illustrated in FIG. 3. ρ of this equation represents blood density.

Aortic pulse wave velocity (V_{pw}) has been shown to be inversely associated with aortic compliance (Lehmann, E. D. Lancet 1999;354(9178):528-529). Increased V_{pw} has also been shown to be an independent predictor of both all-cause and cardiovascular mortality clinically in hemodialysis patients (Blacher et al. Circulation 1999;99:2434-2439). In addition, V_{pw} has also been shown to increase with atherosclerotic disease (Hopins et al. Lancet 1994;343(8911):1447; Blacher et al. Hypertension 1999;35(5): 1111-1117; Lehmann et al. Hypertension 1998;32(3):565-569). Further, in Marfan syndrome patients, beta blocker therapy has been shown to decrease aortic V_{pw} (Groenink et al. Am J Cardiol 1998;82(2):203-208).

Thus, measurement of these parameters, and in particular V_{pw} via the modified esophageal Doppler monitor of the present invention provides a useful method for assessing aortic compliance and/or distensibility, important risk factors in patients with aortic dissections and aortic aneurysms. Subjects at risk for this include, but are not limited to, those with a recent history of trauma, diabetes, renal failure, Marfan's syndrome, a congenital abnormality of connective tissue that can lead to aortic aneurysms and dissections, and atherosclerotic disease characterized by a build-up lipid and cholesterol laden plaques within blood vessel walls.

In this method, the aortic pulse wave velocity is measured in a subject by inserting into the esophagus of the subject the esophageal probe of the modified esophageal Doppler monitor of the present invention. The time between peak velocities of aortic blood flow is detected as each transducer and the aortic pulse wave velocity is calculated as shown supra. The calculated aortic pulse wave velocity in the subject can then be used to assess aortic compliance and/or distensibility. Increased aortic pulse wave velocity in the subject as compared to normal controls is indicative of decreased aortic compliance and/or distensibility.

Assessment of aortic compliance in a subject via measurement of aortic pulse wave velocity using the modified esophageal Doppler monitor of the present invention can also be used to aide in diagnosis of atherosclerotic disease. An increase in aortic pulse wave velocity or a decrease in aortic compliance in a subject as compared to aortic pulse wave velocity or aortic compliance of normal controls is indicative of atherosclerotic disease.

By “normal controls” as used herein it is meant a healthy human with an aortic pulse wave velocity ranging from about 3 to 10 M/S (Bulpitt et al. J Am Geriatr Soc 1999;47(6):657-663; Rogers et al. J Am Coll Cardiol 2001;38:1123-9). Higher velocities may be observed in healthy older individuals as V_{pw} normally increase with age (Bulpitt et al. J Am Geriatr Soc 1999;47(6):657-663; Rogers et al. J Am Coll Cardiol 2001;38:1123-9).

The modified esophageal Doppler monitor of the present invention also provides a useful means for assessing the efficacy of a therapeutic agent for atherosclerotic disease. Efficacy of a therapeutic agent for atherosclerotic disease in a subject can be assessed by first determining aortic compliance in the subject. This is determined by measurement of aortic pulse wave velocity in the subject using the modified esophageal Doppler monitor as described herein. The subject is then administered the agent for a period of time believed to be sufficient for the agent to be therapeutically effective. Following this treatment period aortic compliance in the subject is reassessed. An increase in compliance of the aorta as measured by a decrease in aortic pulse wave velocity in the subject following administration of the therapeutic agent as compared to aortic pulse wave velocity measured in the subject prior to administration of the therapeutic agent is indicative of therapeutic efficacy of the agent. No change in aortic compliance and aortic pulse wave velocity or a decrease in aortic compliance as measured by an increase in aortic pulse wave velocity in the subject following administration of the therapeutic agent as compared to aortic pulse wave velocity measured in the subject prior to administration of the therapeutic agent is indicative of the agent not being therapeutically effective. Assessing the therapeutic efficacy of an agent via the method of the present invention is particularly useful for anti-hypertensive agents such as beta-blockers.

1. A modified esophageal Doppler monitor capable of measuring time rate change of pressure within the aorta and aortic pulse wave velocity as well as cardiac output, afterload, preload, peak velocity and acceleration of aortic blood flow, said modified esophageal Doppler monitor comprising an esophageal probe with two ultrasonic Doppler transducers separated by a known distance.

2. An esophageal Doppler monitor capable of measuring time rate change of pressure within the aorta and aortic pulse wave velocity as well as cardiac output, afterload, preload, peak velocity and acceleration of aortic blood flow, said esophageal Doppler monitor comprising:
(a) a hollow esophageal stethoscope having an upper and a lower end;
(b) a first transducer which measures frequency shift associated with a change in velocity within the aorta, said first transducer mounted at the lower end of the hollow esophageal stethoscope;
(c) a second transducer which measures frequency shift associated with a change in velocity within the aorta, said second transducer mounted in the hollow esophageal stethoscope at a selected distance between the upper and lower end of the stethoscope; and
(d) a means connected to the first and second transducers for calculating time rate change of pressure within the aorta and aortic pulse wave velocity from the frequency shifts measured by each transducer.

3. A method for measuring aortic pulse wave velocity in a subject comprising inserting into the esophagus of a subject the esophageal probe of the modified esophageal Doppler monitor of claim 1; measuring time between peak velocities detected as each transducer; and calculating the aortic pulse wave velocity in the subject.

4. A method for assessing aortic compliance or distensibility in a subject comprising inserting into the esophagus of a subject the esophageal probe of the modified esophageal Doppler monitor of claim 1; and determining aortic wave pulse velocity and time rate change of pressure within the aorta (aortic dP/dt) in the subject so that compliance or distensibility of the aorta in the subject can be assessed.

5. A method for diagnosing atherosclerotic disease in a subject comprising assessing aortic compliance in the subject in accordance with claim 4 wherein a decrease in aortic compliance in the subject as compared to aortic compliance of controls is indicative of atherosclerotic disease.

6. A method of assessing efficacy of a therapeutic agent for atherosclerotic disease in a subject comprising:
(a) assessing aortic compliance in a subject in accordance with the method of claim 4;
(b) administering a therapeutic agent for a selected time adequate to be therapeutically effective to the subject; and
(c) reassessing aortic compliance in the subject in accordance with the method of claim 4 wherein an increase in compliance of the aorta following administration of the therapeutic agent is indicative of therapeutic efficacy.

7. The method of claim 6 wherein the therapeutic agent is an anti-hypertensive agent.
